(19) United States
(12) Patent Application Publication
(10) Pub. No.: US 2006/0142349 A1
(43) Pub. Date: Jun. 29, 2006

(54) Methods of modulating the activities of alpha-7 nicotinic acetylcholine receptor

(75) Inventors: Raymond S. Hurst, Waterford, CT (US); Vincent E. Groppi, Kalamazoo, MI (US); David W. Piotrowski, Waterford, CT (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 11/274,215

(22) Filed: Nov. 15, 2005

Related U.S. Application Data
(60) Provisional application No. 60/639,090, filed on Dec. 23, 2004.

(51) Int. Cl. A61K 31/44; A61K 31/433; A61K 31/42

(52) U.S. Cl. 514/344; 514/362; 514/378

(57) ABSTRACT

The present invention provides methods for identifying compounds useful in modulating α7 nicotinic acetylcholine receptors. The invention also provides compounds that prevent, suppress or inhibit desensitization of an α7 nicotinic acetylcholine receptor, and which resensitize such receptors. Pharmaceutical compositions, and methods of treatment, particularly in regard of neurological diseases are also described.

The present application claims priority under 35 USC section 119 of U.S. provisional application 60/639,090, filed Dec. 23, 2004, and which is incorporated by reference in its entirety, as if fully set forth herein

### FIELD OF THE INVENTION

The present invention relates to methods of identifying compounds useful for modulating an α7 nicotinic acetylcholine receptor. In particular, the present invention relates to compounds that prevent, suppress or inhibit desensitization of an α7 nicotinic acetylcholine receptor and/or compounds that resensitize an α7 nicotinic acetylcholine receptor. The present invention further relates to methods of preventing or treating diseases using compounds identified according to the methods described herein, and pharmaceutical compositions containing such compounds.

### BACKGROUND OF THE INVENTION

Nicotinic acetylcholine receptors define diverse groups of neurotransmitter receptors present in tissues, such as muscle, ganglia and the central nervous system (CNS). The natural activating ligand for the receptors is acetylcholine, but the receptors are also sensitive to the drug nicotine (see Alkondon & Albuquerque, Progress in Brain Research 145: 109-120, 2004). Generally speaking, there are two basic types of nicotinic acetylcholine receptors. The first consists of heteropentameric receptors made of one αsubtype, such as α2, α3, α4, or α6, and one β subtype, such as β2 or β4 (note that β3 and α5 subtypes may also be involved in forming such receptors).

The second type of nicotinic acetylcholine receptor consists of homopentameric receptors composed of α7, α8 or α9 subtypes. See Francis et al., Mol. Pharm. 60: 71-79 (2001), and U.S. Pat. No. 6,693,172. Other subtypes, such as α10, may also be involved in forming nicotinic acetylcholine receptors. See Elgoyhen et al., 10^{th} Neuropharm Conf. (2000).

The α7 nicotinic acetylcholine receptor is a ligand-gated ion channel highly permeable

Activation of the α7 nicotinic acetylcholine receptor may alleviate, treat or prevent many diseases and conditions. These diseases and conditions include cognitive dysfunction, neurodegenerative diseases, such as Alzhimer's disease, Down's syndrome, Parkinson's disease and glaucoma. Activation of the α7 nicotinic acetylcholine receptor may also prevent or alleviate attention deficithyperactivity disorders, anxiety, obsessive-compulsive disorder, drug addition, schizophrenic disorders, and other diseases or conditions exhibiting or caused by neurotoxicity, such as neurotoxicity induced by ethanol. See Kem W R, Behav. Brain Res. 113: 169-181; Leonard et al., Schizophr. Bull. 22: 431-445 (1996); De Fiebre et al., Alcohol 31: 149-153 (2003). Thus, compounds that modulate the activities of the α7 nicotinic acetylcholine receptor have many potential therapeutic applications. See Mullen et al., J. Med. Chem. 43: 4045-4050 (2000).

An α7 nicotinic acetylcholine receptor can be activated by the binding of an agonist. Numerous agonists of the α7 nicotinic acetylcholine receptor have been identified, including nonselective agonists, such as nicotine, and selective agonists, such as 3-[2,4-dimethoxybenzylidene]anabaseine (DMXB), De Fiebre et al., Alcohol 31: 149-153 (2003) and (−)-Spiro[1-azabicyclo[2.2.2]octane-3,5′-oxazolidin-2′-one, Mullen et al., J. Med. Chem. 43: 4045-4050 (2000). Additional examples of such agonists can be found in International Patent Application Publication Nos. WO96/06098, WO97/30998, WO99/03859. In addition to agonists of the α7 nicotinic acetylcholine receptor, other compounds that positively modulate the activities of agonists have also been identified. These compounds may increase the potency of an agonist or the maximal response elicited by an agonist. Examples of such compounds can be found in U.S. Pat. Nos. 6,479,510, 6,277,870 and 6,492,385, U.S. Patent Application, Publication No. 2003.0236287A1 and International Patent Application Publication No. WO01/32620.

Although calcium channels composed of the α7 nicotinic acetylcholine receptor subunits are highly permeable to calcium ions, prolonging exposure of an α7 nicotinic acetylcholine receptor to an agonist often results in rapid desensitization of the α7 nicotinic acetylcholine receptor. Consequently, activation of an α7 nicotinic acetylcholine receptor by an agonist is often temporary. Prevention of this desensitization (as well as resensitization of an inactivated α7 nicotinic acetylcholine receptor) represents a critical issue in developing compound that inhibits, suppresses or prevents desensitization of an α7 nicotinic acetylcholine receptor. Compounds identified according to the practice of the present invention (i.e. that resensitize or prevent desensitization of the α7 nicotinic acetylcholine receptor) may be administered in combination with other compounds that have therapeutic effects on the receptor, such as agonists, and also allosteric modulators of the receptor including those modulators that affect agonist activity (see, for example, U.S. Pat. No. 6,277,870).

In a further preferred embodiment of the invention, individual compounds are identified that have more than one useful effect on the α7 nicotinic acetylcholine receptor, for example that the individual compounds are both positive allosteric modulators (for example, as disclosed in U.S. Patent Publication 2003/0236287) and resensitizers.

In a preferred example of the invention, there is provided the method of (i) contacting a cell expressing an α7 nicotinic acetylcholine receptor with an agonist to desensitize the receptor, (ii) contacting the cell with an effective amount of a candidate compound, and (iii) determining whether the desensitized receptor is resensitized by the candidate compound.

The present invention further provides compounds identified using the methods described herein, and that are capable of resensitizing an α7 nicotinic acetylcholine receptor. Examples of such compounds include, but are not limited to, the following:
N-(5-chloro-2,4-dimethoxyphenyl)-N′-(5-methylisoxazol-3-yl)urea
N-(2,4-dimethoxy-5-methylphenyl)-N′-[5-(trifluoromethyl)-1,3,4-thiadiazol-2-yl]urea
N-(4-ethoxy-2-nitrophenyl)-N′-[3-(trifluoromethyl)isoxazol-5-yl]urea
N-(5-chloro-2,4-dimethoxyphenyl)-N′-(3-methylisoxazol-5-yl)urea
N-[2-(2-furyl)-4-methoxyphenyl]-N′-[5-(trifluoromethyl)-1,3,4-thiadiazol-2-yl]urea
N-(5-bromo-2,4-dimethoxyphenyl)-N′-[3-(trifluoromethyl)isoxazol-5-yl]urea
N-(5-chloro-2,4-dimethoxyphenyl)-N′-[5-(trifluoromethyl)isoxazol-3-yl]urea
N-[4-ethoxy-2-(2-furyl)phenyl]-N′-[5-(trifluoromethyl)-1,3,4-thiadiazol-2-yl]urea
N-(4-methoxy-2-nitrophenyl)-N′-[5-(trifluoromethyl)-1,3,4-thiadiazol-2-yl]urea
N-[4-methoxy-2-(1,3-oxazol-2-yl)phenyl]-N′-[5-(trifluoromethyl)-1,3,4-thiadiazol-2-yl]urea
N-(4-ethoxy-2-nitrophenyl)-N′-[5-(trifluoromethyl)isoxazol-3-yl]urea
N-[2-methoxy-4-(2-methoxyethoxy)phenyl]-N′-[5-(trifluoromethyl)-1,3,4-thiadiazol-2-yl]urea
   α7 nicotinic acetylcholine receptor. The present invention also describes methods of resensitizing an α7 nicotinic acetylcholine receptor which has been desensitized. These methods may be carried out in vitro. They may be also carried out in vivo by administration of a compound capable of suppressing, inhibiting or preventing desensitization, and/or capable of resensitizing an α7 nicotinic acetylcholine receptor which has been desensitized, to a subject in need of treatment therewith.

As aforementioned, the methods described in the present invention are useful for preventing, treating or alleviating various diseases and conditions. These diseases and conditions include, without limitation, cognitive and attention deficit symptoms of Alzheimer's disease, neurodegeneration associated with diseases such as Alzheimer's disease, pre-senile dementia (mild cognitive impairment), senile dementia, schizophrenia or psychosis and cognitive deficits associated therewith, attention deficit disorder, attention deficit hyperactivity disorder (ADHD), mood and affective disorders, amylotrophic lateral sclerosis, borderline personality disorder, traumatic brain injury, behavioral and cognitive problems associated with brain tumors, AIDS dementia complex, dementia associated with Down's syndrome, dementia associated with Lewy Bodies, Huntington's disease, depression, general anxiety disorder, age-related macular degeneration, Parkinson's disease, tardive dyskinesia, Pick's disease, post traumatic stress disorder, dysregulation of food intake including bulemia and anorexia nervosa, withdrawal symptoms associated with smoking cessation and dependent drug cessation, Gilles de la Tourette's Syndrome, glaucoma, neurodegeneration associated with glaucoma, or symptoms associated with pain.

Activation of an α7 nicotinic acetylcholine receptor may also lead to a decrease in levels of TNF-α, which in turn provides symptomatic relief from diseases or conditions, including, but not limited to, any one or more or a combination of the following: inflammation; pain; cancer; or diabetes. Types of inflammation and/or pain that are to be treated include, but are not limited to, any one or more of the following: rheumatoid arthritis; rheumatoid spondylitis; muscle degeneration; osteoporosis; osteoarthritis; psoriasis; contact dermatitis; bone resorption diseases; atherosclerosis; Paget's disease; uveititis; gouty arthritis; inflammatory bowel disease; adult respiratory distress syndrome (ARDS); Crohn's disease; rhinitis; ulcerative colitis; anaphylaxis; asthma; Reiter's syndrome; tissue rejection of a graft; burns, and wounds in general including from surgery), bone fracture healing, ischemic heart disease; and stable angina pectoris. Thus, the compounds identified using the methods described in the present invention are useful for treating, preventing and alleviating various diseases or conditions described above.

The present invention also describes pharmaceutical compositions containing compounds capable of resensitizing an α7 nicotinic acetylcholine receptor, and/or capable of suppressing, inhibiting or preventing desensitization of an α7 nicotinic acetylcholine receptor, and methods of preparing and administering such pharmaceutical compositions.

Finally, it is generally known to one practiced in the art that the agonist-evoked activity of alpha-7 nicotinic acetylcholine receptors can be influenced by allosteric modulators in one of two ways. The first is to increase agonist potency; this class of allosteric modulator has been termed “allosterically potentiating ligand” (e.g., Schrattenholz et al., 1996). The second is to increase the maximal response evoked by an agonist; this class of positive modulator has been termed “efficacy enhancer” by Gurely and Lanthorn (e.g., Eisele et al., 1993; Quik et al., 1997; Gurley and Lanthorn, 2001). The method described in the present invention is different from those described in the prior art in that it specifically identifies compounds that resensitize alpha-7 nicotinic acetylcholine receptors and/or compounds that slow the process of receptor desensitization. This novel and additional property is not predicted in the prior art. For example, a compound previously identified as an efficacy enhancer, 5-hydroxyindole (Gurley and Lanthom, 2001) does not resensitize alpha-7 nicotinic acetylcholine receptors and it does not alter the rate of receptor desensitization. (See also example, see Eisele J L, Bertrand S, Galzi J L, Devillers-Thiery A, Changeux J P, Bertrand D. Chimaeric nicotinic-serotonergic receptor combines distinct ligand binding and channel specificities. Nature. 1993 Dec. 2;366(6454):479-83; Gurley D and Lanthorn T. 2001. U.S. Pat. No. 6,277,870 B1; Quik M, Philie J, Choremis J. Modulation of alpha7 nicotinic receptor-mediated calcium influx by nicotinic agonists. Mol Pharmacol. 1997. 51:499-506; and Schraftenholz A, Pereira E F, Roth U, Weber K H, Albuquerque E X, Maelicke A. Agonist responses of neuronal nicotinic acetylcholine receptors are potentiated by a novel class of allosterically acting ligands. Mol Pharmacol. 1996 January;49(1):1-6).

However, according to the practice of the present invention, it has also been specifically disclosed compounds (named in IUPAC form) also evidence the “efficacy enhancer” property.

An additional compound identified according to the practice of the present invention is

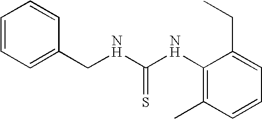

### BRIEF DESCRIPTION OF FIGURES AND DRAWINGS

FIG. 1 illustrates the desensitization of an α7 nicotinic acetylcholine receptor when exposed to Compound 1, an agonist of the α7 nicotinic acetylcholine receptor. In the presence of Compound 1, Compound 2 resensitizes the α7 nicotinic acetylcholine receptor.

FIG. 2 illustrates the desensitization of an α7 nicotinic acetylcholine receptor when exposed to an agonist of the α7 nicotinic acetylcholine receptor. Addition of 5-hydroxyindole did not resensitize the desensitized receptor.

FIG. 3 illustrates the inhibition by Compound 2 of the desensitization of α7 nicotinic acetylcholine receptor by acetylcholine, acting as agonist of the receptor, when the α7 nicotinic acetylcholine receptor was first exposed to Compound 2.

FIG. 4 illustrates that exposure of an α7 nicotinic acetylcholine receptor to each of Compounds 2 to 15 inhibited desensitization by acetylcholine, while 5-hydroxyindole, a modulator of the α7 nicotinic acetylcholine receptor, had no such effect.

### DETAILED DESCRIPTION OF THE INVENTION

The present invention describes methods of identifying a compound that modulates the activities of an α7 nicotinic acetylcholine receptor. In particular, the methods described herein are able to identify a compound capable of resensitizing a desensitized α7 nicotinic another embodiment of the present invention is a method of identifying a compound that modulates the activities of an α7 nicotinic acetylcholine receptor, comprising (i) exposing the α7 nicotinic acetylcholine receptor to a sufficient amount of a candidate compound, (ii) exposing the receptor to an agonist in the presence of the candidate compound, and (iii) measuring one or more activities of the α7 nicotinic acetylcholine receptor in the presence and absence of the candidate compound.

Various methods may be used to desensitize an α7 nicotinic acetylcholine receptor. One preferred method is exposure of an α7 nicotinic acetylcholine receptor to a sufficient amount of an agonist of the α7 nicotinic acetylcholine receptor.

As used herein, the terms “desensitize” and “desensitization” shall encompass, but not be limited to, partial desensitization. Similarly, the term “resensitize” or “resensitization” shall encompass partial resensitization. It should also be understood that, according to the practice of the invention, the terms may be used interchangeably, that is, when a compound is a “resensitizer”, it is often capable of preventing desensitization, and a compound that prevents desensitization is often a resensitizer, thus the terms are not intended to be mutually exclusive, and the assays whereinby such compounds are detected may not distinguish between such effects, especially since the net physiological result achieved may be the same or effectively the same.

Additionally, it should be understood that when an assay involves multiple steps, such as for the addition of reagents capable of binding to the α7 nicotinic acetylcholine receptor, or displacing a compound from said receptor, whether at an agonist binding site, an allosteric modulator binding site, or a resensitization site, as may be the case, the steps may be performed sequentially or simulatenously, as is generally apparent from the assay described, or as those skilled in the art would, of course, generally appreciate.

Desensitization of an α7 nicotinic acetylcholine receptor can, of course, also be accomplished by contacting a cell expressing an α7 nicotinic acetylcholine receptor with a sufficient amount of an agonist of the α7 nicotinic acetylcholine receptor.

A further embodiment of the present invention is a method of identifying a compound that modulates the activities of an α7 nicotinic acetylcholine receptor, comprising (i) contacting a cell expressing the α7 nicotinic acetylcholine receptor with a sufficient amount of a bacterial, yeast or mammalian expression system. Prefreably, a mammalian expression system may be used. A transgenic animal, such as a transgenic mouse or rat, may be created to express an α7 nicotinic acetylcholine receptor. The α7 nicotinic acetylcholine receptor may be isolated and purified from such cells, tissues or animals using standard methods routinely employed in the field to isolate proteins or peptides. See e.g. Scopes et al. Protein Purification: Principles and Practice (1996). The cells may be eukaryotic or prokaryotic cells.

Another embodiment of the invention is a method of identifying a compound that modulates the activities of an α7 nicotinic acetylcholine receptor, comprising (i) contacting a cell expressing the α7 nicotinic acetylcholine receptor with a sufficient amount of a candidate compound, (ii) contacting the cell with a desensitizing agent in the presence of the candidate compound, and (iii) measuring the activities of the α7 nicotinic acetylcholine receptor.

As described herein, the desensitizing compounds may be selected based on their abilities to desensitize an α7 nicotinic acetylcholine receptor. Such compounds may interact directly with the α7 nicotinic acetylcholine receptor. They may also affect the activities of the α7 nicotinic acetylcholine receptor without direct interaction, but through signal cascades. Preferred embodiments of such sensitizing compounds are agonists, including, without limitation, partial agonists, of an α7 nicotinic acetylcholine receptor. A more preferred embodiment is a selective agonist of an α7 nicotinic acetylcholine receptor.

Examples of agonists of the nicotinic acetylcholine receptor include the nonselective agonist, nicotine. Other examples of agonists of an α7 nicotinic acetylcholine receptor can be found in International Patent Application Publiction Nos. WO96/06098, WO97/30998, WO99/03859, and U.S. Patent Application Publication No. 2003/0236287A1. Additional compounds capable of desensitizing an α7 nicotinic acetylcholine receptor may be identified by a person skilled in the art using known techniques. For example, agonists of an α7 nicotinic acetylcholine receptor may be identified using the cell-based, calcium flux assay FLIPR. A description of such an assay can be found, for example, in the International Patent Application Publication No. WO00/73431.

A cell expressing an α7 nicotinic acetylcholine receptor for use in the methods described herein can be obtained from various sources. It can be isolated from tissue culture. compound, and (iii) measuring the activities of the α7 nicotinic acetylcholine receptor in the presence of the candidate compound.

One embodiment of the present invention uses eukaryotic cells, preferably mammalian cells, and more preferably human cells. A variety of cells can be obtained commercially from sources such as the American Tissue Culture Collection (herein “ATCC”). Alternatively, cells such as embryonic stem cells, may be isolated or obtained from natural sources. See Whittemore et al. Int. J. Dev. Neurosci. 11:755-64 (1993). Cells may also exist in tissues or organ preparations, preferably from a mammalian brain or central nervous system, Cells may be, without limitation, from rat, cat, horse, mouse, hamster, chicken, sheep, goat, pig, cow, rabbit, monkey and human. Cells so obtained can be further expanded and harvested from tissue culture, as routinely practiced in the field. See e.g. Bernice M. Martin, Tissue Culture Techniques (Birkhauser Verlag A G, 1994). Examples of cells that may be used in the present invention include, but are not limited to, CHO cells, PC-12 cells, K-177 cells, SH-SY5Y cells, TE-671 cells, HEK293 cells and Xenopus oocytes. See e.g. Rogers et al. Protein Expr Purif. 2(2-3): 108-16 (1991); Fitch et al. PNAS 100: 4909-4914 (2003).

Cells derived from a tissue or organ preparation can be purified or mixed with other components or cells. In one embodiment, isolated tissue, an organ, or fragments or portions of such tissue or organ, derived from an animal, preferably derived from a mammalian central nervous system is used, as long as such tissue or organ displays the activities of the α7 nicotinic acetylcholine receptor. An example of such tissue or organ is, without limitation, a slice of brain from a mammal or a particular section of a mammalian brain. Accordingly, another embodiment of the present invention is (i) contacting tissue or an organ displaying the acitivites of an α7 nicotinic acetylcholine receptor with a sufficient amount of a desensitizing compound to sensitize the α7 nicotinic acetylcholine receptor, (ii) contacting the tissue or organ with a candidate compound, and (iii) measuring the activities of the α7 nicotinic acetylcholine receptor.

Yet another preferred embodiment is a method of identifying a compound capable of suppressing, inhibiting or preventing desensitization of an α7 nicotinic acetylcholine receptor, comprising (i) mixing a cell expressing an α7 nicotinic acetylcholine receptor with a candidate compound, (ii) adding at least one agonist of the α7 nicotinic acetylcholine receptor to the cell including, but not limited to, deletions, substititons, and additions, can be made to an α7 nicotinic acetylcholine receptor, as long as such variations do not eliminate the activities of the α7 nicotinic acetylcholine receptor, or such variations do not prevent the performance of the methods described herein. Certain mutated forms of the α7 nicotinic acetylcholine receptor can be found in the U.S. Pat. No. 6,693,172 and International Patent Application Publication No. WO00/73431. See also Broide et al. Mol. Pharm. 61: 695-705 (2002).

To obtain a cell expressing an α7 nicotinic acetylcholine receptor, a standard approach is construction of an expression vector containing exogenously inserted DNA encoding an α7 nicotinic acetylcholine receptor and transfection of the expression vector into a selected host cell. As described above, DNA encoding an α7 nicotinic acetylcholine receptor may be obtained using different techniques, such as chemical synthesis, and DNA purification from a cell carrying such DNA. Such DNA may then be inserted into an expression vector using standard cloning techniques. See e.g. Sambrook et al., in Molecular Cloning: A Laboratory Manual. Cold Spring Harbor Laboratory Press, NY, Vol. 1, 2, 3 (1989). Vectors, such as pGEX, pBluescript, pET, pPAC, pGEM1 and pCMV-GST, may be used to construct such an expression vector, which often contains necessary elements to permit transcription and/or translation of an exogenously inserted DNA into a polypeptide in a suitable host cell. An expression vector so constructed is then transfected or transformed into a host cell, preferably a eukaryotic cell and more preferably a mammalian cell. Once in the host cell, the expression vector may integrate into the host chromosomal DNA or replicate independently of the host chromosomal DNA.

To desensitize an α7 nicotinic acetylcholine receptor, cells expressing the α7 nicotinic acetylcholine receptor may be exposed to a sufficient amount of an agonist of the α7 nicotinic acetylcholine receptor. See e.g. Radcliffe et al. J. Neurosci. 18: 7075-7083 (1998), Alkondon et al. Neuropharmacology 39: 2726-2739 (2000). The level of desensitization generally depends on the amount of the agonist and duration of the exposure, as achieved by a person skilled in the art.

Desensitization of an α7 nicotinic acetylcholine receptor may be dependent on the concentration of a desensitizing compound. In a preferred method, a sufficient amount of a desensitizing compound is added to saturate the available α7 nicotinic acetylcholine exposure, or sooner. However, the duration of exposure may vary, and a person skilled in the art may change such duration to achieve the optimal results.

To identify a compound capable of resensitizing an α7 nicotinic acetylcholine receptor and/or capable of suppressing, preventing or inhibiting desensitization of an α7 nicotinic acetylcholine receptor, a cell-free system may be used in which an α7 nicotinic acetylcholine receptor is reconstituted in vitro. See Briley & Changeux, Int Rev Neurobiol. 20:31-63 (1977). One embodiment of the present invention comprises the step of isolating α7-nicotinic acetylcholine receptor proteins and reconstructing an α7 nicotinic acetylcholine receptor in a cell-free system. See e.g. Sobel et al., Eur J Biochem. 80: 215-224 (1977); McNamee & Ochoa, Neuroscience 7: 2305-2319 (1982). Various materials may be used to constitute such a cell-free system. See e.g. Baenziger et al. Biophys J. 61: 983-992 (1992).

In a preferred embodiment, the isolated α7 nicotinic acetylcholine receptor proteins are incorporated into a liposome preparation, or other lipid vesicles using lipid components, such as dioleoyl phosphatidyl choline. This allows the construction of an artificial membrane comprising the α7 nicotinic acetylcholine receptor embedded in lipid bilayers. Such a membrane may be used to identify a compound capable of modulating an α7 nicotinic acetylcholine receptor, as described in the present invention. The methods of obtaining or preparing liposomes and other lipid vesicles are well known to a person skilled in the art. See e.g. Gregoriadis, Liposome Technology: Preparation of Liposomes (1984).

Thus, another embodiment of the present invention is a method of identifying a compound capable of resensitizing an α7 nicotinic acetylcholine receptor, comprising (i) desensitizing an α7 nicotinic acetylcholine receptor with an agonist of the α7 nicotinic acetylcholine receptor, (ii) exposing the desensitized α7 to a candidate compound, and (iii) measuring the activities of the α7 nicotinic acetylcholine receptor in the presence of the candidate compound, wherein the α7 nicotinic acetylcholine receptor is in a cell-free system. A further embodiment of the present invention is a method of identifying a compound capable of suppressing or preventing desensitization of an α7 nicotinic acetylcholine receptor, comprising (i) exposing the α7 nicotinic acetylcholine receptor to an agonist in the presence of a candidate compound, and (ii) measuring the activities of the α7 nicotinic acetylcholine receptor, wherein the α7 nicotinic acetylcholine receptor is in a cell-free system.

al. J. Neuroscience 18: 648-657 (1998); Gould et al. Life Sci. 33: 2665-2672 (1983); Fitch et al. PNAS 100: 4909-4914 (2003); Lukas R J J. Neurochem. 46: 1936-1941 (1986); Connolly & Kennedy, Drugs Pharm. Sci. 89: 107-133 (1998); Connolly & Kennedy, J Recept Signal Transduct Res. 21: 191-214 (2001); Velicelebi et al. Methods Enzymol. 279: 20-47 (1999); Bertrand et al. J. Recept. Sig. Transd. Res. 17: 227-242 (1997); Villarroya et al. Methods Mol. Biol. 114: 137-147 (1999). All these references are hereby incorporated by reference.

The methods described above can also be used to identify or screen for multiple compounds capable of modulating the activities of an α7 nicotinic acetylcholine receptor. Thus, another embodiment of the present invention is to screen multiple candidate compounds to identify at least one compound that modulates the activities of an α7 nicotinic acetylcholine receptor. One embodiment of the method comprises (i) selecting multiple candidate compounds, (ii) exposing each candidate compound to at least one cell expressing the α7 nicotinic acetylcholine receptor, and (iii) measuring or comparing the activities of the α7 nicotinic acetylcholine receptor in the presence and absence of each candidate compound. One preferred embodiment is a high throughput screening assay for identifying one or multiple compounds that suppress, inhibit or prevent desensitization of an α7 nicotinic acetylcholine receptor, or resensitize an α7 nicotinic acetylcholine receptor, comprising contacting a cell expressing a desensitized α7 nicotinic acetylcholine receptor with each candidate compound, comparing each candidate compound's ability to modulate the activities of the α7 nicotinic acetylcholine receptor, especially its ability to suppress, inhibit or prevent desensitization of the α7 nicotinic acetylcholine receptor or its ability to resensitize the α7 nicotinic acetylcholine receptor. An even more preferred embodiment is an automated high throughput screening assay.

Using the methods described herein, some compounds capable of resensitizing an α7 nicotinic acetylcholine receptor and/or inhibiting, preventing, or suppressing desensitization of an α7 nicotinic acetylcholine receptor have been identified. Examples of such compounds include, but not are not limited to, the following:
N-(5-chloro-2,4-dimethoxyphenyl)-N′-(5-methylisoxazol-3-yl)urea
N-(2,4-dimethoxy-5-methylphenyl)N′-[5-(trifluoromethyl)-1,3,4-thiadiazol-2-yl]urea
N-(4-ethoxy-2-nitrophenyl)-N′-[3-(trifluoromethyl)isoxazol-5-yl]urea
N-(4-ethoxy-2-nitrophenyl)-N′-[5-(trifluoromethyl)isoxazol-3-yl]urea
N-[2-methoxy-4-(2-methoxyethoxy)phenyl]-N′-[5-(trifluoromethyl)-1,3,4-thiadiazol-2-yl]urea
N-(6-cyanopyridin-3-yl)-N′-(5-fluoro-2,4-dimethoxyphenyl)urea
N-(4-methoxy-2-methylphenyl)-N′-[5-(trifluoromethyl)-1,3,4-thiadiazol-2-yl]urea
or a pharmaceutically acceptable salt thereof.

The preparation, use and administration of the above described compounds is described in U.S. Patent Application Publication No. 20030236287A1.

Accordingly, another embodiment of the present invention is a method of resensitizing an α7 nicotinic acetylcholine receptor in a cell, comprising contacting the cell with an effective amount of at least one compound, whose structure is described above. Yet another embodiment of the present invention is a method of suppressing, inhibiting or preventing desensitization of an α7 nicotinic acetylcholine receptor in a cell, comprising contacting the cell with an effective amount of at least one compound, whose structure is described above.

Stated additionally, compounds identified according to the practice of the present invention positively modulate the α7 nicotinic acetylcholine receptor by slowing receptor desensitization when the receptor is in the continuous presence of an introduced agonist, or the natural agonist acetylcholine, and the agonist is bound. Similarly, compounds identified according to the practice of the present invention facilitate resensitization of a receptor that has already been desensitized by an agonist, whether or not agonist has been discharged or remains bound to the receptor. As a result, according to the practice of the invention, the time average of total agonist-evoked current (when measured over the whole cell) can be increased, and the duration of current at an individual alpha 7 receptor can be increased. In this way, the “response rate” to acetylcholine or another agonist can be increased, meaning the total amount of ions that flow in response to agonist binding is increased. In this regard, the reader is referred to published U.S. patent application Publication Ser. No. 20030236287A1, which describes measuring an increase of the peak agonist evoked current, and also R. Hurst et al., “A novel positive allosteric modulator of the alpha7 neuronal nicotinic acetylcholine receptor: in vitro and in vivo characterization”, The Journal of Neuorscience, v. 25(17), pp. 4396-4405, 2005 which more particularly describes these assays both in the context of whole impairment), senile dementia, schizophrenia or psychosis and cognitive deficits associated therewith, attention deficit disorder, attention deficit hyperactivity disorder (ADHD), mood and affective disorders, amyotrophic lateral sclerosis, borderline personality disorder, traumatic brain injury, behavioral and cognitive problems associated with brain tumors, AIDS dementia complex, dementia associated with Down's syndrome, dementia associated with Lewy Bodies, Huntington's disease, depression, general anxiety disorder, age-related macular degeneration, Parkinson's disease, tardive dyskinesia, Pick's disease, post traumatic stress disorder, dysregulation of food intake including bulemia and anorexia nervosa, withdrawal symptoms associated with smoking cessation and dependant drug cessation, Gilles de la Tourette's Syndrome, glaucoma, neurodegeneration associated with glaucoma, or symptoms associated with pain.

As aforementioned, activation of an α7 nicotinic acetylcholine receptor may also lead to decrease of levels of TNF-α, which in turn provides symptomatic relief from diseases or conditions, including, but not limited to, any one or more or a combination of the following: inflammation; pain; cancer; or diabetes. Types of inflammation and/or pain that are to be treated include, but are not limited to, any one or more of the following: rheumatoid arthritis; rheumatoid spondylitis; muscle degeneration; osteoporosis; osteoarthritis; psoriasis; contact dermatitis; bone resorption diseases; atherosclerosis; Paget's disease; uveititis; gouty arthritis; inflammatory bowel disease; adult respiratory distress syndrome (ARDS); Crohn's disease; rhinitis; ulcerative colitis; anaphylaxis; asthma; Reiter's syndrome; tissue rejection of a graft; ischemia reperfusion injury; brain trauma; stroke; multiple sclerosis; cerebral malaria; sepsis; septic shock; toxic shock syndrome; fever and myalgias due to infection; HIV-1, HIV-2, and HIV-3; cytomegalovirus (CMV); influenza; adenovirus; a herpes virus (including HSV-1, HSV-2); or herpes zoster. Types of cancer that are to be treated include, but are not limited to, any one or more of the following: multiple myeloma; acute and chronic myelogenous leukemia; or cancer-associated cachexia. Finally, activation of an α7 nicotinic acetylcholine receptor may also treat or prevent obesity, bulimia, or obesity-related disease.

Thus, another embodiment of the present invention is a method of suppressing, preventing, or inhibiting desensitization of an α7 nicotinic acetylcholine receptor in a subject in need thereof, comprising administering to the subject an effective amount of a compound that more preferably a human, in need thereof, comprising administering to the subject an effective amount of a compound that resenstizes the α7 nicotinic acetylcholine receptor. The present invention also describes a method of treating, alleviating, or preventing a disease or condition described above, in a subject, preferably a mammal and more preferably a human, in need thereof, comprising administering to the subject an effective amount of a compound that suppresses, prevents or inhibits desensitization of the α7 nicotinic acetylcholine receptor.

In particular, one preferred embodiment of the present invention is a method of treating, alleviating, or preventing a schizophrenic disorder in a subject, preferably a mammal, and more preferably a human, in need thereof, comprising administering to the subject an effective amount of a compound that resensitizes the α7 nicotinic acetylcholine receptor, and a method of treating, alleviating, or preventing a schizophrenic disorder in a subject, preferably a mammal and more preferably a human, in need thereof, comprising administering to the subject an effective amount of a compound that suppresses, prevents, or inhibits desensitization of the α7 nicotinic acetylcholine receptor.

Yet another preferred embodiment is a method of treating, alleviating, or preventing obesity in a subject, preferably a mammal and more preferably a human, in need thereof, comprising administering to the subject an effective amount of a compound that resenstizes the α7 nicotinic acetylcholine receptor, and a method of treating, alleviating, or preventing obesity in a subject, preferably a mammal and more preferably a human, in need thereof, comprising administering to the subject an effective amount of a compound that suppresses, prevents or inhibits desensitization of the α7 nicotinic acetylcholine receptor or a compound that resensitizes the α7 nicotinic acetylcholine receptor.

The compounds described herein, and other compounds which may be identified using the methods described herein, are useful in maintaining the active state of an α7 nicotinic acetylcholine receptor. Accordingly, these compounds are particularly useful when used in combination with other agents that could modulate the activities of an α7 nicotinic acetylcholine receptor, such as an agonist of an α7 nicotinic acetylcholine receptor or an allosteric modulator thereof.

Representative agonist compounds are disclosed in the following published United States patent applications and issued United States patents, U.S. Pat. No. 2002-0,086,871, US
N-[(exo-(4S)-1-azabicyclo[2.2.1]hept-3-yl]furo[3,2-c]pyridine-6-carboxamide;
N-(1-(6-methyl)-azabicyclo[2.2.1]hept-3-yl)furo[3,2-c]pyridine-6-carboxamide;
N-((3R,5R)-1-azabicyclo[3.2.1]oct-3-yl)furo[3,2-c]pyridine-6-carboxamide;
N-(1-azabicyclo[3.2.2]non-3-yl)furo[3,2-c]pyridine-6-carboxamide;
N-(1-azabicyclo[2.2.1]hept-3-yl)-2-methylfuro[2,3-c]pyridine-5-carboxamide;
N-(1-(6-methyl)-azabicyclo[2.2.1]hept-3-yl)-2-methylfuro[2,3-c]pyridine-5-carboxamide;
N-((3R,5R)-1-azabicyclo[3.2.1]oct-3-yl)-2-methylfuro[2,3-c]pyridine-5-carboxamide;
Exo-4(S)—N-(1-azabicyclo[2.2.1]hept-3-yl)-3-methylfuro[2,3-c]pyridine-5-carboxamide;
N-(1-(6-methyl)-azabicyclo[2.2.1]hept-3-yl)-3-methylfuro[2,3-c]pyridine-5-carboxamide;
(exo)-N-[1-Azabicyclo[3.2.1]oct-3-yl]-3-methylfuro[2,3-c]pyridine-5-carboxamide;
(3R,5R)-N-[1-azabicyclo[3.2.1]oct-3-yl]-3-methylfuro[2,3-c]pyridine-5-carboxamide;
Exo-4(S)—N-(1-azabicyclo[2.2.1]hept-3-yl)-3-ethylfuro[2,3-c]pyridine-5-carboxamide;
N-(1-(6-methyl)-azabicyclo[2.2.1]hept-3-yl)-3-ethylfuro[2,3-c]pyridine-5-carboxamide;
N-((3R,5R)-1-azabicyclo[3.2.1]oct-3-yl)-3-ethylfuro[2,3-c]pyridine-5-carboxamide;
N-(1-azabicyclo[2.2.1]hept-3-yl)-thieno[2,3-b]pyridine-5-carboxamide;
N-(1-(6-methyl)-azabicyclo[2.2.1]hept-3-yl)-thieno[2,3-b]pyridine-5-carboxamide;
N-((3R,5R)-1-azabicyclo[3.2.1]oct-3-yl)thieno[2,3-b]pyridine-5-carboxamide;
N-(1-azabicyclo[3.2.2]non-3-yl)-thieno[2,3-b]pyridine-5-carboxamide;
N-(1-azabicyclo[2.2.1]hept-3-yl)-thieno[2,3-b]pyridine-6-carboxamide;
N-(1-(6-methyl)-azabicyclo[2.2.1]hept-3-yl)-thieno[2,3-b]pyridine-6-carboxamide;
N-((3R,5R)-1-azabicyclo[3.2.1]oct-3-yl)-thieno[2,3-b]pyridine-6-carboxamide;
N-(1-azabicyclo[3.2.2]non-3-yl)-thieno[2,3-b]pyridine-6-carboxamide;
N-(1-azabicyclo[2.2.1]hept-3-yl)-thieno[3,2-b]pyridine-5-carboxamide;
N-(1-(6-methyl)-azabicyclo[2.2.1]hept-3-yl)-thieno[3,2-b]pyridine-5-carboxamide;
N-((3R,5R)-1-azabicyclo[3.2.1]oct-3-yl)-thieno[3,2-b]pyridine-5-carboxamide;
N-(1-azabicyclo[3.2.2]non-3-yl)-thieno[3,2-b]pyridine-5-carboxamide;
N-(1-azabicyclo[2.2.1]hept-3-yl)-thieno[3,2-b]pyridine-6-carboxamide;
N-(1-(6-methyl)-azabicyclo[2.2.1]hept-3-yl)-thieno[3,2-b]pyridine-6-carboxamide;
N-(1-(6-methyl)-azabicyclo[2.2.1]hept-3-yl)-thieno[3,2-c]pyridine-6-carboxamide;
N-((3R,5R)-1-azabicyclo[3.2.1]oct-3-yl)-thieno[3,2-c]pyridine-6-carboxamide;
N-(1-azabicyclo[3.2.2]non-3-yl)-thieno[3,2-c]pyridine-6-carboxamide;
N-[exo-(4S)-1-azabicyclo[2.2.1]hept-3-yl]-3-chlorofuro[2,3-c]pyridine-5-carboxamide;
N-(1-(6-methyl)-azabicyclo[2.2.1]hept-3-yl)-3-chlorofuro[2,3-c]pyridine-5-carboxamide;
N-((3R,5R)-1-azabicyclo[3.2.1]oct-3-yl)-3-chlorofuro[2,3-c]pyridine-5-carboxamide;
4-Benzooxazol-2-yl-1,4-diaza-bicyclo[3.2.2]nonane;
2-(1,4-Diaza-bicyclo[3.2.2]non-4-yl)-1-oxa-3-aza-cyclopenta[b]-naphthalene;
4-Benzothiazol-2-yl-1,4-diaza-bicyclo[3.2.2]nonane;
4-(5-Phenyl-benzooxazol-2-yl)-1,4-diaza-bicyclo[3.2.2]nonane;
4-(1H-Benzoimidazol-2-yl)-1,4-diaza-bicyclo[3.2.2]nonane;
4-(6-Phenyl-benzooxazol-2-yl)-1,4-diaza-bicyclo[3.2.2]nonane;
2-(1,4-Diaza-bicyclo[3.2.2]non-4-yl)-3-oxa-1-aza-cyclopenta[a]-naphthalene;
4-(5-Chloro-benzooxazol-2-yl)-1,4-diaza-bicyclo[3.2.2]nonane;
4-(5-Fluoro-benzooxazol-2-yl)-1,4-diaza-bicyclo[3.2.2]nonane;
4-(6-Nitro-benzooxazol-2-yl)-1,4-diaza-bicyclo[3.2.2]nonane;
4-Oxazolo[5,4-b]pyridin-2-yl-1,4-diaza-bicyclo[3.2.2]nonane;
4-Oxazolo[5,4-c]pyridin-2-yl-1,4-diaza-bicyclo[3.2.2]nonane;
4-Oxazolo[4,5-c]pyridin-2-yl-1,4-diaza-bicyclo[3.2.2]nonane;
4-Oxazolo[4,5-b]pyridin-2-yl-1,4-diaza-bicyclo[3.2.2]nonane;
4-(5-Pyridin-3-yl-benzooxazol-2-yl)-1,4-diaza-bicyclo[3.2.2]-nonane;
4-(5-Bromo-benzooxazol-2-yl)-1,4-diaza-bicyclo[3.2.2]nonane;
4-(6-Bromo-oxazolo[5,4-b]pyridin-2-yl)-1,4-diaza-bicyclo[3.2.2]-nonane;
4-(5-Iodo-benzooxazol-2-yl)-1,4-diaza-bicyclo[3.2.2]nonane;
4-(4-Nitro-benzooxazol-2-yl)-1,4-diaza-bicyclo[3.2.2]nonane;
4-(5-Nitro-benzooxazol-2-yl)-1,4-diaza-bicyclo[3.2.2]nonane;
4-(5-Methyl-benzooxazol-2-yl)-1,4-diaza-bicyclo[3.2.2]nonane;
4-(6-Methyl-benzooxazol-2-yl)-1,4-diaza-bicyclo[3.2.2]nonane;
4-(5-Methyl-oxazolo[4,5-b]pyridin-2-yl)-1,4-diaza-bicyclo[3.2.2]nonane;
4-(6-Phenyl-oxazolo[5,4-b]pyridin-2-yl)-1,4-diaza-bicyclo[3.2.2]nonane;
4-[5-(4-Trifluoromethyl-phenyl)-benzooxazol-2-yl]-1,4-diaza-bicyclo[3.2.2]nonane;
4-(6-Bromo-oxazolo[4,5-b]pyridin-2-yl)-1,4-diaza-bicyclo[3.2.2]nonane;
4-(6-Phenyl-oxazolo[4,5-b]pyridin-2-yl)-1,4-diaza-bicyclo[3.2.2]nonane; and
4-(5,7-Dichloro-benzooxazol-2-yl)-1,4-diaza-bicyclo[3.2.2]nonane;
4-(6-bromo-5-methyloxazolo[4,5-b]pyridin-2-yl)-1,4-diaza-bicyclo[3.2.2]nonane;
4-(6-Bromo-5-ethyl-oxazolo[4,5-b]pyridin-2-yl)-1,4-diaza-bicyclo[3.2.2]nonane;
4-(5,6-dimethyloxazolo[4,5-b]pyridin-2-yl)-1,4-diaza-bicyclo[3.2.2]nonane;
4-(6-Methyl-5-ethyl-oxazolo[4,5-b]pyridin-2-yl)-1,4-diaza-bicyclo[3.2.2]nonane;
4-(5-Methyl-6-nitro-oxazolo[4,5-b]pyridin-2-yl)-1,4-diaza-bicyclo[3.2.2]nonane;
2-(1,4-Diaza-bicyclo[3.2.2]non-4-yl)-5-methyl-oxazolo[4,5-b]pyridin-6-ylamine;
4-(6-Fluoro-5-methyl-oxazolo[4,5-b]pyridin-2-yl)-1,4-diaza-bicyclo[3.2.2]nonane;
4-(6-Chloro-5-methyl-oxazolo[4,5-b]pyridin-2-yl)-1,4-diaza-bicyclo[3.2.2]nonane;
4-(6-Chloro-5-ethyl-oxazolo[4,5-b]pyridin-2-yl)-1,4-diaza-bicyclo[3.2.2]nonane;
4-(5-methyl-6-phenyloxazolo[4,5-b]pyridin-2-yl)-1,4-diaza-bicyclo[3.2.2]nonane;
4-(5-methyl-6-phenoxyoxazolo[4,5-b]pyridin-2-yl)-1,4-diaza-bicyclo[3.2.2]nonane;
2-(1,4-diaza-bicyclo[3.2.2]nonan-4-yl)-5-methyloxazolo[4,5-b]pyridine-6-carbonitrile;
4-(6-(2-fluoro-6-methoxyphenyl)oxazolo[5,4-b]pyridin-2-yl)-1,4-diaza-bicyclo[3.2.2]nonane;
4-(6-(1H-pyrazol-4-yl)oxazolo[5,4-b]pyridin-2-yl)-1,4-diaza-bicyclo[3.2.2]nonane;
4-(6-(thiazol-2-yl)oxazolo[5,4-b]pyridin-2-yl)-1,4-diaza-bicyclo[3.2.2]nonane;
4-(6-(oxazol-2-yl)oxazolo[5,4-b]pyridin-2-yl)-1,4-diaza-bicyclo[3.2.2]nonane;
4-(6-(2-fluorophenyl)oxazolo[5,4-b]pyridin-2-yl)-1,4-diaza-bicyclo[3.2.2]nonane;
4-(6-(2-methoxypyridin-3-yl)oxazolo[5,4-b]pyridin-2-yl)-1,4-diaza-bicyclo[3.2.2]nonane;
(2-(2-(1,4-diaza-bicyclo[3.2.2]nonan-4-yl)oxazolo[5,4-b]pyridin-6-yl)phenyl)methanol;
4-(6-(2-fluorophenyl)oxazolo[4,5-b]pyridin-2-yl)-1,4-diaza-bicyclo[3.2.2]nonane;
2-(2-(1,4-diaza-bicyclo[3.2.2]nonan-4-yl)oxazolo[4,5-b]pyridin-6-yl)benzonitrile;
4-(6-(2-fluoro-6-methoxyphenyl)oxazolo[4,5-b]pyridin-2-yl)-1,4-diaza-bicyclo[3.2.2]nonane;
4-(5-Phenethyl-oxazolo[4,5-b]pyridin-2-yl)-1,4-diaza-bicyclo[3.2.2]nonane;
4-(5-Morpholin-4-yl-oxazolo[4,5-b]pyridin-2-yl)-1,4-diaza-bicyclo[3.2.2]nonane;
(R)-(−)-2-(1,4-diazabicyclo[3.2.1]oct-4-yl)[1,3]oxozolo[4,5-b]pyridine,
(R)-(−)-6-chloro-2-(1,4-diazabicyclo[3.2.1]oct-4-yl)[1,3]oxozolo[5,4-b]pyridine,
(R)-(−)-6-bromo-2-(1,4-diazabicyclo[3.2.1]oct-4-yl)[1,3]oxozolo[5,4-b]pyridine,
(R)-(−)2-(1,4-diazabicyclo[3.2.1]oct-4-yl)-5-methyl[1,3]oxazolo[4,5-b]pyridine,
(R)-(−)-6-chloro-2-(1,4-diazabicyclo[3.2.1]oct-4-yl)[1,3]oxazolo[4,5-b]pyridine,
(R)-(−)-6-bromo-2-(1,4-diazabicyclo[3.2.1]oct-4-yl)[1,3]oxazolo[4,5-b]pyridine,
(R)-(−)-2-(1,4-diazabicyclo[3.2.1]oct-4-yl)6-phenyl[1,3]oxazolo[4,5-b]pyridine,
(R)-(−)-2-(1,4-diazabicyclo[3.2.1]oct-4-yl)-6-(2-fluoro-6-methoxyphenyl)[1,3]oxazolo[4,5-b]pyridine,
(R)-(−)-2-(1,4-diazabicyclo[3.2.1]oct-4-yl)-6-(2-fluoro-6-methoxyphenyl)[1,3]oxazolo[5,4-b]pyridine,
(R)-(−)-2-(1,4-diazabicyclo[3.2.1]oct-4-yl)-6-(2-fluoro-6-methylphenyl)[1,3]oxazolo[4,5-b]pyridine,
(R)-(−)-2-(1,4-diazabicyclo[3.2.1]oct-4-yl)-6-(2-fluoro-6-methylphenyl)[1,3]oxazolo[5.4-b]pyridine,
(R)-(−)-2-(1,4-diazabicyclo[3.2.1]oct-4-yl)[1,3]oxazolo[4,5-b]pyridine-6-carbonitrile,
(R)-(−)-2-(1,4-diazabicyclo[3.2.1]oct-4-yl)-6-methyl[1,3]oxazolo[4,5-b]pyridine,
(R)-(−)-2-(1,4-diazabicyclo[3.2.1]oct-4-yl)-6-ethyl[1,3]oxazolo[4,5-b]pyridine,
(R)-(−)-6-Chloro-2-(1,4-diaza-bicyclo[3.2.1]oct-4-yl)-5-methyl-oxazolo[4,5-b]pyridine, (R)-(−)2-(1,4-diaza-bicyclo[3.2.1]oct-4-yl)-5-methyl-oxazolo[4,5-b]pyridine-6-carbonitrile,
(R)-(−)-6-bromo-2-(1,4-diazabicyclo[3.2.1]oct-4-yl)-5-methyl[1,3}-oxazolo[4,5-b]pyridine,
(R)-(−)-2-(1,4-diazabicyclo[3.2.1]oct-4-yl)-6-phenoxy[1,3]oxazolo[5,4-b]pyridine,
(R)-(−)-2-(1,4-diazabicyclo[3.2.1]oct-4-yl)-5,6-dimethyl[1,3]-oxazolo[4,5-b]pyridine.
(S)-(+)-2-(1,4-diazabicyclo[3.2.1]oct-4-yl)[1,3]oxozolo[5,4-b]pyridine,
(S)-(+)-6-chloro-2-(1,4-diazabicyclo[3.2.1]oct-4-yl)[1,3]oxozolo[5,4-b]pyridine,
(S)-(+)-6-bromo-2-(1,4-diazabicyclo[3.2.1]oct-4-yl)[1,3]oxozolo[5,4-b]pyridine,
(S)-(+)-6-chloro-2-(1,4-diazabicyclo[3.2.1]oct-4-yl)[1,3]oxozolo[5,4-b]pyridine, and
   α7 nicotinic acetylcholine receptor, and (ii) contacting the cell with an agonist or modulator of the α7 nicotinic acetylcholine receptor.

Accordingly, the present invention describes a method of treating or preventing a disease or condition in a subject in need thereof, preferably in a mammal, and more preferably in a human, comprising (i) administering to the subject an effective amount of at least one compound that suppresses, inhibits or prevents desensitization of an α7 nicotinic acetylcholine receptor and/or resensitizes an α7 nicotinic acetylcholine receptor and (ii) also administering to the subject an agonist of the α7 nicotinic acetylcholine receptor.

In combination use, the compounds of the present invention may be co-administered simultaneously, or at separate intervals with other agents. When co-administered simultaneously with the other agents, the compounds of the present invention and the other agent(s) can be incorporated into a single pharmaceutical composition. A pharmaceutical composition may comprise one compound of the present invention and an agonist of the α7 nicotinic acetylcholine receptor, as well as other ingredients that have therapeutic effects. Alternatively, when the compounds of the present invention are administered separately from other agents, more than one composition, e.g., one containing a compound of the present invention and the other containing the other agent, can be administered.

One preferred embodiment of the present invention is therefore a method of treating or preventing a schizophrenic disorder in a subject in need thereof, comprising administering to the subject an effective amount of a compound that inhibits or suppresses desensitization of an α7 nicotinic acetylcholine receptor or a compound capable of resensitizing the α7 nicotinic acetylcholine receptor, with an agonist of the α7 nicotinic acetylcholine receptor. An even more preferred embodiment further comprises also administering to the subject one ore more antipsychotic drugs, as the term is generally recognized.

In addition to agents that modulate the activities of an α7 nicotinic acetylcholine receptor, the compounds of the present invention may also be administered in combination with other agents that may not directly modulate the activities of an α7 nicotinic acetylcholine receptor, such as certain antibacterial and antiviral agents for treating infection; certain anticancer agents and/or antiemetic agents for treating cancer; certain agents for treating diabetes, or certain agents for treating obesity. Other agents that may be co-administered pharmaceutical composition suitable for treating, preventing or alleviating a disease or condition in a subject in need thereof, preferably a mammal or more preferably a human, comprising an effective amount of at least one compound that suppresses, inhibits or prevents desensitization of an α7 nicotinic acetylcholine receptor and/or capable of resensitizing an α7 nicotinic acetylcholine receptor, and a pharmaceutically acceptable carrier. According to one very preferred embodiment, the pharmaceutical composition may further comprise at least one agonist of the α7 nicotinic acetylcholine receptor, and/or other modulators of the α7 nicotinic acetylcholine receptor.

As used herein, the term “carrier” includes acceptable diluents, excipients, adjuvants, vehicles, solubilization aids, viscosity modifiers, preservatives, and other agents well known to the artisan for providing favorable properties in the final pharmaceutical composition.

The pharmaceutical composition may be formulated into different forms depending on the delivery systems to be used. By way of example, the pharmaceutical compositions of the present invention may be formulated to be delivered orally as tablets or capsules, as a nasal spray or aerosol for inhalation, as an ingestable solution, or parenterally, in which the composition is formulated in an injectable form, for delivery, by, for example, an intravenous, intramuscular or subcutaneous route. Alternatively, the formulation may be designed to be delivered by both routes.

The dosage and dose rate of the compounds identified in the present invention effective for treating or preventing a disease or condition will depend on a variety of factors, such as the nature of the disease or condition, the size of the patient, the goal of the treatment, the nature of the pathology to be treated, the specific pharmaceutical composition used, and the observations and conclusions of the treating physician.

For example, where the dosage form is oral, e.g., a tablet or capsule, suitable dosage levels can generally be between about 0.1 μg/kg and about 50.0 mg/kg body weight per day, preferably between about 1.0 μg/kg and about 5.0 mg/kg body weight per day, more preferably between about 10.0 μg/kg and about 1.0 mg/kg of body weight per day, and most typically between about 20.0 μg/kg and about 0.5 mg/kg of body weight per day of the active ingredient.

The number of times per day that a dose is administered will depend upon such

### EXAMPLE 1

### Preparation of Cells Expressing an α7 Nicotinic Acetylcholine Receptor

Sprague-Dawley rats (postnatal day 3) were decapitated and brains were removed and placed in ice cold artificial cerebrospinal fluid (ACSF) containing NaCl 130 mM, NaHCO₃ 26 mM, NaH₂PO4 1.25 mM, KCl 3 mM, CaCl₂ 1 mM, MgCl₂ 5 mM and glucose 10 mM. Hippocampal regions were gently removed and cut into small pieces. The tissues were then placed in Hibernate-A media containing 1 mg/ml papain for 60 min at 35° C. After digestion, the tissues were washed in Hibernate-A media obtained from BrainBits, LLC, Springfield, Ill. (catalog no. HA) and transferred to a 50 ml conical tube containing 2 ml trituration medium, which is made of Hibernate-A medium, with 2% B27 serum-free supplement obtained from Invitrogen, Carlsbad, Calif.

Neurons were dissociated by trituration. Hippocampi were slowly triturated until the media became cloudy with cells, and only a few larger tissue pieces remained. Cells were then purified over a Nycoprep gradient in accord with the methods described in Brewer G J, Mol. Chem. Neuropathol. 31(2): 171-86 (1997). Briefly, the Nycoprep gradient was made in four 1 ml steps of 35, 25, 20, and 15% Nycoprep in trituration medium. The cell suspension was then centrifuged, and the fraction containing the cells of interest were plated onto poly-D-lysine/laminin coated coverslips at a density of 300-700 cells/mm. The cells were left at room temperature, in the hood, for 1 hr to allow the cells to adhere to the coverslips. The coverslips were then transferred to tissue culture plates containing warmed culture medium, which was made of Neurobasal-A medium, obtained from Invitrogen (catalog no. 10888-022) with B27 serum-free supplement (2%), L-glutamine (0.5 mM), 100 U/mI penicillin, 100 mg/ml streptomycin, and 0.25 mg/ml Fungizone. Cells were maintained in a humidified incubator at 37° C. and 6% CO2 for 1-2 weeks. The medium was changed after 24 hours, and approximately every three days thereafter.

### EXAMPLE 2

### Measurement of the Activities of an α7 Nicotinic Acetylcholine Receptor

The activity of an α7 nicotinic acetylcholine receptor was measured using an electrophysiological method. Currents were recorded from individual neurons using the whole 7-nitro-quinoxaline 2,3-(1H, 4H)-dione (CNQX), a glutamate receptor antagonist at a concentration of 5 micromolar, and tetrodotoxin (TTX), a Na+ channel blocker, at a concentration of 0.5 micromolar were included in the bath to diminish spontaneous synaptic activity.

Experiments which used acetylcholine as the agonist had 1 mM atropine sulphate in all solutions. Candidate compounds were delivered by a multibarrel fast perfusion exchange system obtained from Warner Instruments of Hamden, Conn. Patch pipettes were made from borosilicate capillary glass, and filled with an internal pipette solution consisting of CsCH₃SO₃, 126 mM; CsCl, 10 mM; NaCl, 4 mM; MgCl₂, 1 mM; CaCl₂, 0.5 mM; EGTA, 5 mM; HEPES, 10 mM; ATP-Mg2+, 3 mM; GTP-Na, 0.3 mM; phosphocreatine, 4 mM at pH 7.2 and 280 mOsm. The resistance of the patch pipettes when filled with internal solution ranged from 3 to 6 MOhm. Experiments were carried out at room temperature.

### EXAMPLE 3

### Identification of a Resensitizer of α7 Nicotinic Acetylcholine Receptors

Candidate compounds were tested for their activities in resensitizing an α7 nicotinic acetylcholine receptor as follows: an agonist of the α7 nicotinic acetylcholine receptor was applied to a cell expressing the α7 nicotinic acetylcholine receptor for 3 minutes. The concentration of the agonist was such that the α7 nicotinic acetylcholine receptors were first activated and then fully desensitized in the continued presence of the agonist. 3 μM of the agonist was used in this experiment, although 100 nM of an agonist should be sufficient to fully desensitize the receptor. The agonist used in these experiments was (N-[(3R)-1-azabicyclo[2.2.2]oct-3-yl]-4-chlorobenzamide hydrochloride (Compound 1), whose preparation is described in PCT Application No. WO0285901.

In the presence of the agonist, a candidate compound was added such that the concentration of the agonist remained the same during the experiment. The compound that resensitized an α7 nicotinic acetylcholine receptor was identified by its ability to evoke the α-7 nicotonic receptor-mediated response in the continued presence of the agonist, as measured using an electrophysiological method. In this example, the activity of the α7 nicotinic acetylcholine receptor was measured using the whole cell patch clamp technique. See Fenwick et al., J. Physiol. 331: 577-597 (1982.). Other methods such as calcium imaging N-(5-chloro-2,4-dimethoxyphenyl)-N′-(5-methylisoxazol-3-yl)urea, is applied in the continued presence of nicotine. Addition of nicotine should result in an initial inward current that rapidly declines back to baseline due to receptor desensitization. Addition of Compound 2 should result in a large persistent inward current that is readily reversed upon washout.

### EXAMPLE 5

### Comparison of A Desensitizer and A Modulator of the α7 Nicotinic Acetylcholine Receptor

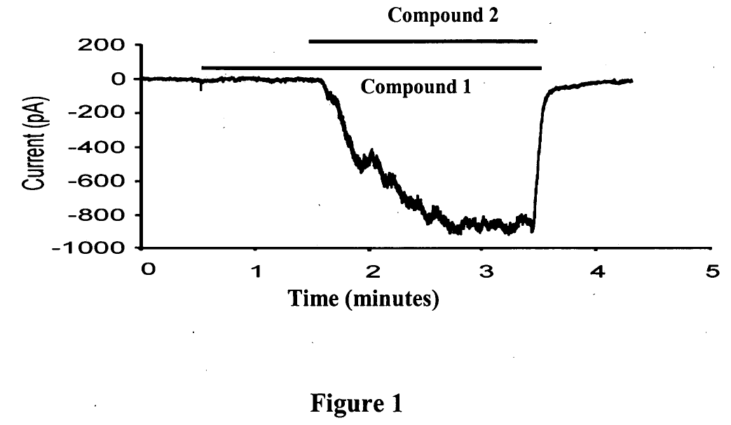

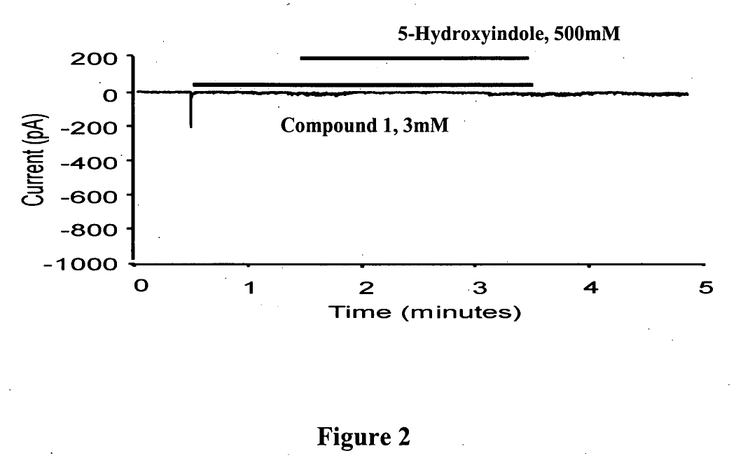

Cells were prepared in accordance with the methods described in Example 1 and exposed to Compound 1, a selective agonist of the α7 nicotinic acetylcholine receptor, continuously for 3 minutes. After 60 seconds of continuous exposure to the agonist, Compound 2 or 5-hydroxyindole were applied in the continued presence of Compound 1. As shown in FIG. 1 and FIG. 2 wherein the dark lines represent the duration that the receptor was exposed to a compound, addition of the agonist resulted in an initial inward current that rapidly declined back to baseline due to receptor desensitization. Addition of Compound 2 resulted in a large persistent inward current that was readily reversed upon washout. Addition of 5-hydroxyindole has no significant effect on the inward current.

### EXAMPLE 6

### Inhibition of Desensitization of An α7 Nicotinic Acetylcholine Receptor

Cells were prepared in accord with the methods described in Example 1 and exposed to various candidate compounds, including the following:

Compound 2: N-(5-chloro-2,4-dimethoxyphenyl)-N′-(5-methylisoxazol-3-yl)urea

Compound 3: N-(2,4-dimethoxy-5-methylphenyl)-N′-[5-(trifluoromethyl)-1,3,4-thiadiazol-2-yl]urea

Compound 4: N-(4-ethoxy-2-nitrophenyl)-N′-[3-(trifluoromethyl)isoxazol-5-yl]urea

Compound 5: N-(5-chloro-2,4-dimethoxyphenyl)-N′-(3-methylisoxazol-5-yl)urea

Compound 6: N-[2-(2-furyl)-4-methoxyphenyl]-N′-[5-(trifluoromethyl)-1,3,4-thiadiazol-2-yl]urea

Compound 7: N-(5-bromo-2,4-dimethoxyphenyl)-N′-[3-(trifluoromethyl)isoxazol-5-yl]urea

Compound 8: N-(5-chloro-2,4-dimethoxyphenyl)-N′-[5-(trifluoromethyl)isoxazol-3-yl]urea

Compound 13: N-[2-methoxy-4-(2-methoxyethoxy)phenyl]-N′-[5-(trifluoromethyl)-1,3,4-thiadiazol-2-yl]urea

Compound 14: N-(6-cyanopyridin-3-yl)-N′-(5-fluoro-2,4-dimethoxyphenyl)urea

Compound 15: N-(4-methoxy-2-methylphenyl)-N′-[5-(trifluoromethyl)-1,3,4-thiadiazol-2-yl]urea

The preparation of these compounds can be found in published U.S. Patent Application Publication No. 20030236287.

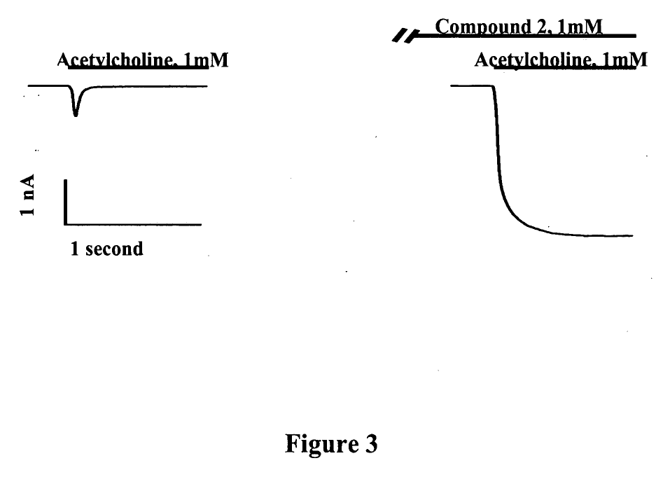

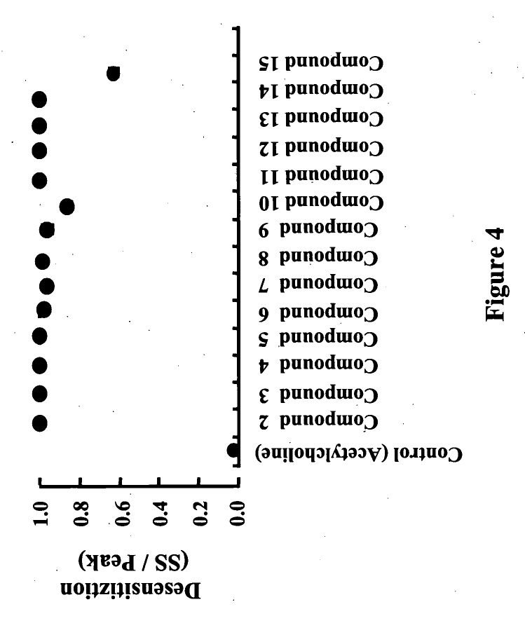

Each compound was applied continuously for 5 to 10 minutes. After approximately 30 seconds of continuous exposure to a candidate compound, acetylcholine (1 mM), an agonist of the α7 nicotinic acetylcholine receptor, was applied for 1 second in the continuous presence of the candidate compound. This procedure was repeated once every minute for a minimum of 5 applications of the agonist acetylcholine. Acetylcholine was washed away between each application, such that the α7 nicotinic acetylcholine receptor was exposed to the agonist for 1 second once every minute, while simultaneously being exposed continuously to each candidate compound. As shown in FIG. 3, exposure of an α7 nicotinic acetylcholine receptor to compound 2 resulted in significant inhibition or slowing of receptor desensitization during the 1 second periods of agonist application. Furthermore, as shown in FIG. 4, 5-hydroxyindole did not affect the desensitization kinetics of acetylcholine-induced ion currents. In contrast, a marked difference in kinetic properties was observed in cells exposed to each of compounds 2 to 15, indicating inhibition of desensitization of the α7 nicotinic acetylcholine receptor by each of these compounds.

### EXAMPLE 7

### Effect of a Candidate Compound on Schizophrenia

The following protocol is used to determine the effect of a candidate compound identified herein in patients suffering schizophrenic disorders.

A randomized, double-blind, placebo controlled study is conducted. Approximately 100 patients, both men and women between the ages of 20 and 50, with a diagnosis of schizophrenia, are recruited for participation in the study.

Patients are randomized for administration of the candidate compound in the amount of 0.1 mg/day to 1000 mg/day, or a placebo for twelve weeks. Prior to randomization, Various mammalian α7 nicotinic acetylcholine receptor constructs, when properly expressed in cells, can be used according to the practice of the present invention. For example, full length wild-type human receptor is very useful, and is described in U.S. Pat. Nos. 5,837,489 and 6,664,375.

However, it is generally recognized that α7 nicotinic acetylcholine receptor is difficult to express on cell membranes in useful quantites. Therefore a special double mutant construct is highly preferred, see U.S. Pat. No. 6,693, 172 to Vincent Groppi et al., which is incorporated by reference herein in its entirety. In this double mutant construct, position 207 is changed from threonine to proline, and position 218 is changed from cysteine to serine (these positions correspond to positions 230 and 241 of Seq ID NO 8 in the U.S. Pat. No. 5,837,489 patent owing to cleavage of a signal peptide). This construct is very well expressed in cells.

Additional α7 nicotinic acetylcholine receptor constructs may be used, although they are less preferred since, lacking all of the alpha7 structure, the results may not compare accurately to requirements needed for treatment of patients. Examples include extracellular domains of alpha 7, preferably from human, but also from other mammals, and any of the well known chimeric constructs known as alpha7/5-HT3, a preferred example of which has the amino terminal, extracellular, or ligand-binding domain of human neuronal nicotinic acetylcholine receptor (minus the signal peptide) fused to the transmembrane and intracellular domains of the 5-hydroxytryptamine(5-HT3) receptor, which is a serotinon receptor. In a preferred example, the 5-HT3 domains are murine, and the utility of such constructs includes to facilitate high levels of expression in recombinant cells, a problem which is also well solved in the human “double mutant” construct of the '172 patent. See also Elsele et al., nature, v. 366 pp. 479-483, 1993. Of course, membrane-vesicles, synthetic or natural cell derived, may also be used to present receptor construct, and alpha7 constructs bound to surfaces may also be used, although such embodiments are, again, less preferred.

### EXAMPLE 9

### Additional Methodology Useful in the Practice of the Invention

Additional background material and various assays useful in the practice of the invention are described in the Appendix attached hereto, as Appendix pages A-1 to A-41 which includes 8 Figures.

1. A method of identifying a compound that suppresses, inhibits, or prevents desensitization of an α7 nicotinic acetylcholine receptor, comprising the steps of (i) exposing a cell expressing the α7 nicotinic acetylcholine receptor to a candidate compound, (ii) exposing the cell to an agonist of the α7 nicotinic acetylcholine receptor that can cause desensitization thereof, and (iii) determining whether the candidate compound prevents or inhibits desensitization of the α7 nicotinic acetylcholine receptor.

2. A method of identifying a compound capable of resensitizing an α7 nicotinic acetylcholine receptor, comprising (i) contacting a cell expressing the α7 nicotinic acetylcholine receptor with an agonist of the α7 nicotinic acetylcholine receptor in a sufficient amount to desensitize the receptor; (ii) contacting the cell with a candidate compound in the presence of the agonist, and (iii) determining whether the desensitized receptor is resensitized by the candidate compound.

3. A pharmaceutical composition useful for treating, preventing or alleviating a disease or condition, comprising an effective amount of a compound that suppresses, inhibits, or prevents desensitization of an α7 nicotinic acetylcholine receptor, or that is capable of resensitizing said receptor, and a pharmaceutically acceptable carrier.

4. A method of suppressing, preventing, or inhibiting desensitization of an α7 nicotinic acetylcholine receptor, or of resensitizing said receptor, in a subject in need thereof, comprising administering to the subject an effective amount of a pharmaceutical composition according to claim 3.

5. The method of claim 4, wherein the compound of said pharmaceutical formulation is selected from a group consisting of:
N-(5-chloro-2,4-dimethoxyphenyl)-N′-(5-methylisoxazol-3-yl)urea
N-(2,4-dimethoxy-5-methylphenyl)-N′-[5-(trifluoromethyl)-1,3,4-thiadiazol-2-yl]urea
N-[4-methoxy-2-(1,3-oxazol-2-yl)phenyl]-N′-[5-(trifluoromethyl)-1,3,4-thiadiazol-2-yl]urea
N-(4-ethoxy-2-nitrophenyl)-N′-[5-(trifluoromethyl)isoxazol-3-yl]urea
N-[2-methoxy-4-(2-methoxyethoxy)phenyl]-N′-[5-(trifluoromethyl)-1,3,4-thiadiazol-2-yl]urea
N-(6-cyanopyridin-3-yl)-N′-(5-fluoro-2,4-dimethoxyphenyl)urea
N-(4-methoxy-2-methylphenyl)-N′-[5-(trifluoromethyl)-1,3,4-thiadiazol-2-yl]urea and pharmaceutically acceptable salt thereof.

6. The method of claim 4, wherein the disease or condition is a schizophrenic or other psychotic disorder, obesity, or a neurodegenerative disease.

7. A method of treating or preventing a disease or condition in a subject in need thereof, comprising administering to the subject an effective amount of (1) a first compound in conjunction with (2) at least one agonist of an α7 nicotinic acetylcholine receptor, wherein said first compound prevents or inhibits desensitization of the α7 nicotinic acetylcholine receptor to the agonist, or causes resensitization of the receptor to the agonist.

8. The method of claim 7, wherein the disease or condition is a schizophrenic or other psychotic disorder, obesity, or a neurodegenerative disease.
